(19) United States
(12) Patent Application Publication
(10) Pub. No.: US 2007/0062517 A1
(43) Pub. Date: Mar. 22, 2007

(54) Oral drug delivery system and methods of use thereof

(76) Inventor: Nicholas P. Barker, Southborough, MA (US)

Correspondence Address: CLARK & ELBING LLP, 101 FEDERAL STREET, BOSTON, MA, 02110, US

(21) Appl. No.: 11/434,372

(22) Filed: May 15, 2006

Related U.S. Application Data
(60) Provisional application No. 60/682,047, filed on May 18, 2005.

(51) Int. Cl. A61M 11/00

(52) U.S. Cl. 128/200.14

(57) ABSTRACT

The invention features devices and methods for delivering drugs to the oral cavity in order to treat mucositis, stomatitis, and other disorders of the oral cavity.

### CROSS-REFERENCE TO RELATED APPLICATIONS

This application claims the benefit of U.S. Provisional Application No. 60/682,047, filed May 18, 2005, which is hereby incorporated by reference.

### BACKGROUND OF THE INVENTION

The invention relates to the field of treatment and prevention of disease.

Oral mucositis is characterized by the breakdown of the oral mucosa, which results in erythema, ulcerations, and pain in the oral cavity. Mucositis often arises as a complication of antineoplastic therapy such as cancer chemotherapy or radiation therapy. The painful ulcerative lesions of mucositis can cause patients to restrict their oral intake of food and liquids; as a result, they lose weight and suffer from dehydration. Severe mucositis can necessitate the de-escalation or the complete interruption of antineoplastic therapy.

In addition to causing pain, mucositis lesions are also sites of secondary infections, acting as portals of entry for endogenous oral microorganisms; this is a particularly serious concern in patients who are immunocompromised. Mucositis is therefore a significant risk factor for chronic debilitating local infections (e.g., yeast infections) as well as life-threatening systemic infection (septicemia). Patients with mucositis and neutropenia have a relative risk of septicemia that is at least four times greater than that of individuals without mucositis. Mucositis is further described in U.S. Pat. Nos. 6,841,578 and 6,663,850 and in U.S. patent application Ser. No. 10/434,752, each of which is hereby incorporated by reference.

Another common and painful oral disorder is aphthous stomatitis. Approximately 10% of the population suffers from these mouth sores at one time or another. The etiology of aphthous stomatitis is not well understood, although the ulcers of this disorder tend to be associated with stress and minor injury to the inside of the mouth.

Standard therapy for these disorders is predominantly palliative. In the case of mucositis, analgesics such as lidocaine may be administered topically, and narcotics and antibiotics may be administered systemically. For aphthous stomatitis, topical application of steroids may provide relief for some patients. However, no satisfactory treatments are currently available for either condition. There is thus a need for an effective means to treat and prevent mucositis, aphthous stomatitis, and similar diseases and disorders of the oral cavity.

### SUMMARY OF THE INVENTION

In one aspect, the invention features a liquid medication delivery apparatus that includes a vial for containing liquid medication and a pump assembly detachably attached to the vial, such pump assembly including a dip tube with an opening positioned inside the vial, a moveable spray arm with a nozzle, and a means for metering and expelling medication from the vial through the opening in the dip tube and out the spray arm nozzle.

In particular embodiments of the above aspect, the vial includes an inner well at its base in which medication may accumulate, with the opening of the dip tube positioned inside the well. In one embodiment, the well is in the shape of a frustum.

In particular embodiments of the above aspect, the apparatus possesses a low center of gravity, restoring itself to an upright position when oriented within 15 degrees of an upright position and released on a horizontal surface.

In particular embodiments of the above aspect, the dip tube opening is notched to allow the dip tube to contact the vial directly without creating a seal in the process. In one embodiment, the notch is v-shaped.

In particular embodiments of the above aspect, the apparatus is used to treat a disorder of the oral cavity. Oral disorders that can be treated by the invention include mucositis (e.g., minor or severe mucositis), stomatitis, aphthous stomatitis, candidiasis, gingivitis, periodontal disease, periocoronitis, periodontitis, xerostomia (dry mouth), abscesses, cold sores, halitosis, viral lesions within the oral cavity (e.g., herpes zoster), fungal infection within the oral cavity (e.g., candidiasis associated with steroid treatment), traumatic injury to the oral cavity, and sequelae of dental surgery, including tooth extraction. For example, the apparatus can be used to treat the throat (e.g., the back of the throat), the inner cheeks, the buccal sulcus, the tongue, the sublingual region, the gingivae, the soft palate, the hard palate, the esophagus, or any other region of the oral cavity. The apparatus can be used to direct medication to any desired region or regions of the oral cavity. In one embodiment, the oral disorder is induced by antineoplastic therapy (for example, chemotherapy, e.g., cisplatin treatment, or radiation therapy, e.g., using cobalt radiation or linear accelerator radiation).

In particular embodiments of the above aspect, the apparatus is used to bypass a patient's taste receptors by spraying one or more regions of the oral cavity that are devoid of taste receptors. For example, any medication with an unpleasant taste, e.g., an anti-fungal medication, can be administered, e.g., to the back of the throat, using an apparatus of the invention.

In particular embodiments of the above aspect, the apparatus contains liquid medication, e.g., any prescription or over-the-counter medication. In one embodiment, the apparatus is filled to capacity; in another embodiment, the apparatus is filled partially. In still another embodiment, the medication includes a pharmaceutically acceptable carrier. In yet another embodiment, the medication includes purified polypeptide such as a trefoil peptide. In one embodiment, the trefoil peptide is intestinal trefoil factor (ITF), or an analog or biologically active fragment of ITF, in dimeric form. In one embodiment, human ITF is used. In another embodiment, the trefoil peptide is spasmolytic polypeptide (SP) or pS2, or an analog or biologically active fragment of spasmolytic polypeptide or pS2. In still another embodiment, the trefoil peptide is hITF₁₅₋₇₃, hITF₂₅₋₆₂, hITF₂₂₋₆₂, hITF₂₁₋₆₂, hITF₂₅₋₇₀, hITF₂₂₋₇₀, hITF₂₅₋₇₂, hITF₂₂₋₇₂, hITF₂₁₋₇₂, hITF₂₅₋₇₃, hITF₂₂₋₇₃, hITF₂₁₋₇₃, or EA-hITF₁₅₋₇₃; descriptions of these fragments may be found in U.S. patent application Ser. No. 10/698,572, which is hereby incorporated by reference.

In particular embodiments of the above aspect, the means for expelling medication includes a spray actuator.

In particular embodiments of the above aspect, the means for expelling medication includes an electronic component. For example, the pump assembly may be actuated by electronic rather than manual means, or by a combination of both electronic and manual means.

In particular embodiments of the above aspect, a liquid medication-containing apparatus dispenses an effective dosage of the medication through the spray arm nozzle upon deployment of the pump assembly. The apparatus may be used to dispense any effective dose volume, e.g., 10 μl, 50 μl, 100 μl, 200 μl, 500 μl, 1 ml, 2 ml, 5 ml, or 10 ml. The apparatus may operate as a unit dose, multiple unit dose, or multidose delivery system.

In particular embodiments of the above aspect, the pump assembly, once primed, remains primed.

In particular embodiments of the above aspect, the moveable spray arm is shorter than 5 centimeters (cm). In other embodiments, the moveable spray arm is 5 cm or longer.

In particular embodiments of the above aspect, the vial is compatible with more than one pump assembly. In one embodiment, the volume of medication expelled in each actuation may be changed by detaching one pump assembly and attaching another pump assembly; each pump assembly fits the general description given above but can differ in the volume of the metering chamber within the pump.

In particular embodiments of the above aspect, the apparatus requires less than 100 Newtons of force to actuate. In other embodiments, the apparatus requires less than 10 Newtons of force to actuate. In still other embodiments, the apparatus requires less than 1 Newton of force to actuate.

In particular embodiments of the above aspect, a liquid medication-containing apparatus, upon actuation, produces a spray of medication in which no greater than 1% of the droplets of medication in the spray are smaller than 10 microns in aerodynamic diameter. In other embodiments, the apparatus contains medication consisting of a solution, suspension, or emulsion. In still other embodiments, the medication includes a mucoadhesive agent. By “mucoadhesive” is meant a composition, including polymers, oligomers, or mixtures, that can adhere to mucous membranes, generally via non-covalent interactions such as hydrogen bonding and van der Waals forces (see, for example, Tabor, J. Colloid Interface Sci. 58:2-13, 1977, and Good, J. Colloid Interface Sci. 59:398-419, 1977, each of which is hereby incorporated by reference). The mucoadhesive agent may contain cellulose, starch, or other compounds. Typical mucoadhesive agents are Carbopols, pectins, alginates, chitosan, oligosaccharides, polysaccharides, gellan, carrageenan, xanthan gum, gum Arabic, Tamarind gum, dextrans, cellulose, derivatives of cellulose, polyether polymers, polyether oligomers, fatty acids, fatty alcohols, fatty amides, polyhydric alcohols, polyether compounds, polyacrylamides, poly(vinyl pyrrolidone), poly(methacrylic acid), poly(acrylic acid), and Carbomers; other mucoadhesive agents can be used as well. In one embodiment, the medication additionally includes a surfactant. For example, the surfactant could include a sorbitan ester, such as Tween or Span compounds. In yet other embodiments, the medication contains an antimicrobial preservative. Effective antimicrobial preservatives include methyl paraben and propyl paraben.

In particular embodiments of the above aspect, the apparatus retains sterility integrity during use. An apparatus engineered in this manner prevents regression of micro-organisms from the tip of the nozzle back into the actuator or vial. In one embodiment, the apparatus is manufactured as a sterile product, obviating the need for an antimicrobial preservative.

In particular embodiments of the above aspect, the apparatus additionally includes materials that have low sorption capability. In one embodiment, the vial is composed of glass. In another embodiment, the vial is composed of plastic.

In a second aspect, the invention features a liquid medication delivery apparatus that includes a vial for containing liquid medication and a pump assembly detachably attached to the vial, such pump assembly including an intake opening positioned below the vial, a moveable spray arm with a nozzle, and a means for metering and expelling medication from the vial into the pump assembly via an orifice, through the metering chamber and out the spray arm nozzle. The apparatus of this aspect does not require a dip tube due to its inverted orientation.

### BRIEF DESCRIPTION OF THE DRAWINGS

FIG. 1A is a side view of an assembled liquid medication delivery apparatus of the invention.

FIG. 1B is a closer view of the dip tube and the well of the invention.

FIG. 2A is an exploded view of the apparatus of FIG. 1.

FIG. 2B is a further exploded view of the screw top assembly of FIG. 2A.

FIG. 2C is a side view showing the interior of the pump of FIG. 2B.

FIG. 2D is a bottom view of the spray actuator of FIG. 2A.

### DESCRIPTION OF A PARTICULAR EMBODIMENT

There now follows a description of a particular embodiment of the invention.

Structure

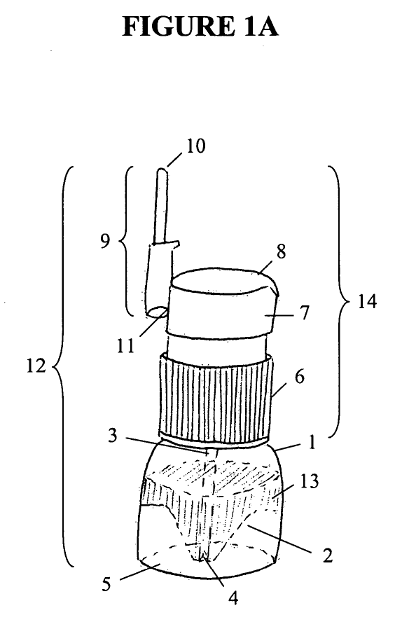

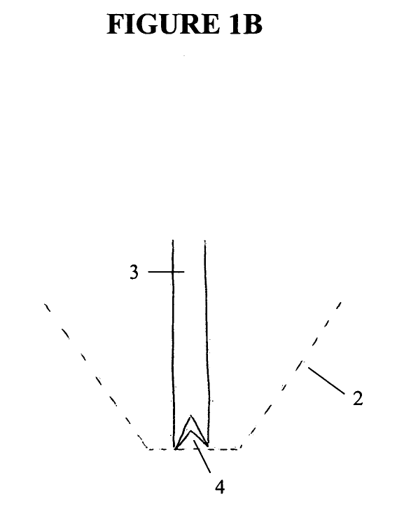

Referring to FIG. 1A, liquid medication delivery apparatus (12) includes vial (1) containing well (2) in base (5) in which liquid medication (13) may be placed. Apparatus (12) also includes pump assembly (14), which includes dip tube (3), screw top assembly (6), spray actuator (7), and spray arm (9). Dip tube (3) includes dip tube opening (4) and is part of screw top assembly (6). Spray actuator (7) also connects to screw top assembly (6) and includes spray actuator indentation (8). Spray arm (9) is connected to spray actuator (7) and contains spray arm nozzle (10) through which medication (13) may be expelled. Spray arm (9) is connected to spray actuator (7) via swivel (11).

Referring to FIG. 1B, dip tube opening (4) includes a notch.

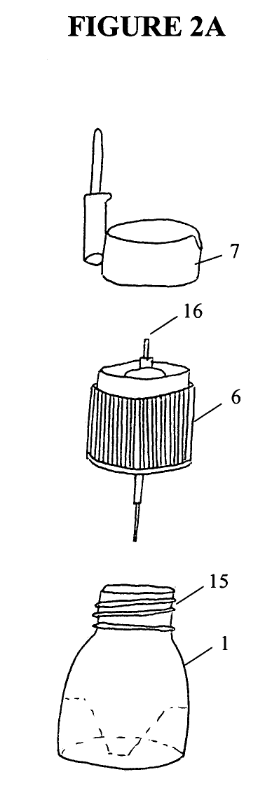

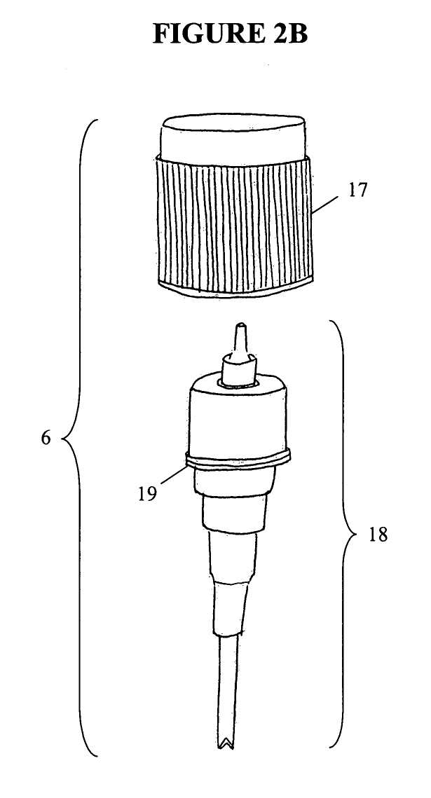

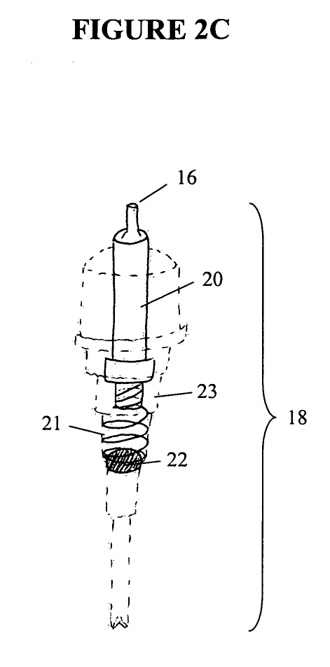

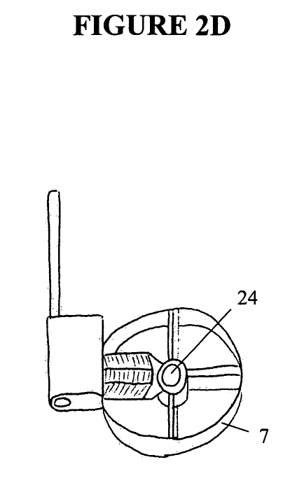

Referring to FIG. 2A, screw top assembly (6) connects to vial (1) via screw threads (15). Spray actuator (7) connects to screw top assembly (6) via actuator connector (16).

Referring to FIG. 2B, screw top assembly (6) consists of screw top casing (17) and pump (18), which snap together for assembly. Pump (18) includes dip tube (3) and gasket (19).

Referring to FIG. 2C, pump (18) includes plunger (20), spring (21), valve (22), and constant volume metering chamber (23). Spring (21) is wrapped around a region of plunger (20) (as shown). Plunger (20) includes actuator connector (16).

Referring to FIG. 2D, spray actuator (7) contains spray actuator opening (24), which connects to actuator connector (16).

Manufacture

Apparatus (12) is constructed from plastics or other materials that possess low sorption capability. The plastic components of apparatus (12) are fabricated using conventional multicavity or single cavity plastic molding. Visual inspection systems may be used to assure the quality and dimension tolerances of the components. Pump assembly (14), including gasket (19), plunger (20), spring (21), valve (22), constant volume metering chamber (23), screw top casing (17), spray actuator (7), and spray arm (9), is then assembled in clean conditions, e.g., a Class 10,000 HEPA filtered-air environment. As appropriate, apparatus (12) may be sterilized by gamma irradiation in an aseptic processing step.

Vial (1) is made by standard molded glass extrusion fabrication techniques. Dimensions of vial (1) are engineered to the length of dip tube (3). Screw threads (15), included on vial (1), are compatible with screw top assembly (6) of pump assembly (14). As appropriate, vial (1) is sterilized by dry heat sterilization or gamma irradiation.

Medication (13) is formulated according to pharmaceutical techniques known to skilled artisans. For example, an emulsion, suspension, or liposomal formulation is prepared by a high-shear mixing process. Typically, the formulation is a solution, in which drug is dispersed and dissolved in a vehicle using a means of agitation. Subsequently, additional excipients, such as those to modify pH, osmolality, viscosity or mucoadhesive properties, are then incorporated at the appropriate concentration. As part of an aseptic processing step, the formulation is filtered through, e.g., a 0.2 micron filter to remove foreign materials. The formulation may be, though it need not be, aseptic processed, and it may include antimicrobial preservative.

As an additional part of an aseptic processing step, medication (13) is filled volumetrically into vial (1) by means of a metering pump. Vial (1) is then closed with pump assembly (14) and subjected to quality control tests.

Operation

Apparatus (12) provides therapeutic doses of liquid medication to the oral cavity of a patient suffering from mucositis induced by antineoplastic therapy. Medication (13) includes purified recombinant dimeric human intestinal trefoil peptide spanning residues 15-73 (hITF₁₅₋₇₃) expressed in Pichia pastoris. Medication (13) also includes a mucoadhesive agent to improve retention of hITF₁₅₋₇₃ on the oral mucosal surface. This agent is effective in adhering to oral mucosal surfaces, but it does not bind or interact with hITF₁₅₋₇₃, nor does it affect cell motogenesis. In addition, medication (13) contains a surfactant to increase the wettability of the mucoadhesive and thus its retention capability. Inclusion of an antimicrobial preservative in formulation of medication (13) enables its use as a multidose system over a period of many days such that apparatus (12) may be used before the expiration of medication (13) contained therein.

Apparatus (12) delivers 100 μl of liquid medication (13), which contains a fixed dose of 4 mg of hITF₁₅₋₇₃, in each actuation cycle. At each scheduled administration time, apparatus (12) is actuated three times, with the patient aiming at a different region of the oral mucosal surfaces each time, or alternatively at the same region each time, and medication (13) is administered to the oral mucosal surfaces. Because saliva naturally dilutes the therapeutic, the patient administers medication (13) multiple times per day. A standard course of treatment includes eight administrations (three actuations each) spread throughout each day.

The patient actuates apparatus (12) by holding such apparatus upright near the mouth, orienting spray arm (9) such that spray arm nozzle (10) is aimed approximately 3 cm from the oral surface, placing a thumb on spray actuator indentation (8), and pressing, thereby causing spray actuator (7) to be depressed. Depression of spray actuator (7) causes plunger (20) inside pump (18) to be depressed, causing spring (21) to compress and valve (22) to form a seal inside pump (18). The seal prevents air or medication (13) from exiting pump (18) via dip tube opening (4), instead causing medication (13) to pass through spray actuator opening (24) and exit from spray arm nozzle (10). In order to maintain the seal and prevent device leakage, the top rim of vial (1) is designed to be flat and smooth in order to ensure a tight fit against gasket (19) upon assembly and thereby prevent device leakage. Medication (13) is metered via constant volume metering chamber (23) and expelled in a spray plume onto the oral surface. The patient then releases spray actuator (7), which returns to its original position. Release of spray actuator (7) results in the release of plunger (20), causing valve (22) to move and medication (13) to be drawn into pump (18) through dip tube opening (4).

Actuation of apparatus (12) requires minimal force, making apparatus (12) particularly amenable to use by the sick or elderly. The patient may use a mirror to guide the orientation and placement of spray arm (9).

Prior to initial administration of medication (13), apparatus (12) must first be primed. Priming is performed by depressing spray actuator (7) and releasing as described above. Five to six cycles of priming are performed before initial therapeutic use of liquid medication delivery apparatus (12). Subsequently, no further priming is necessary as long as liquid medication delivery apparatus (12) remains upright so that dip tube opening (4) remains immersed in medication (13) when activated.

To optimize performance, apparatus (12) should be maintained in an upright position at all times in order to keep medication (13) in well (2) and above the level of dip tube opening (4). The patient aims apparatus (12) by orienting spray arm (9) appropriately while keeping apparatus (12) upright. When maintained in this manner, nearly 100% of medication (13) in vial (1) is available for therapeutic administration. Well (2) is a particularly important feature when medication (13) is expensive or difficult to obtain and overage must be minimized. As an example, in order to dispense 3 100 μl doses of medication (13) 8 times per day, approximately 3.0 ml medication (13) is required; this results in an overage of only 0.6 ml, in comparison to a 1.5-2.0 ml overage in a conventional medication delivery apparatus.

Apparatus (12) delivers a highly reproducible volume of liquid medication (13) in each actuation cycle. The spray plume produced in each actuation cycle consists of droplets of low momentum, resulting in a gentle spray that does not further irritate the oral mucosal surfaces due to impact. The low-momentum spray results from the geometry of the spray arm (9) and spray arm nozzle (10), as well as from the design of pump (18). Medication may be administered at cool temperatures (2-8° C.) to aid soothing.

The spray plume produced by apparatus (12) causes medication (13) to be evenly distributed over the targeted mucosal surface. This is a particularly useful feature for treatment of stomatitis. Because spray arm (9) is moveable, any accessible region of the mucosal surface is easily targeted.

Ordinarily, the patient does not inhale during administration of medication (13). However, if the patient does inhale, a small amount of medication (13) may get into the patient's lungs. In order to minimize lung exposure, no more than 5% of the number of droplets of medication (13) in the spray plume produced by each actuation cycle are of respirable size (approximately 10 microns in aerodynamic diameter or smaller). 10% of the droplets of medication (13) are smaller than 16 microns in aerodynamic diameter. 50% of the droplets of medication (13) are smaller than 49 microns in aerodynamic diameter. 90% of the droplets of medication (13) are smaller than 163 microns in aerodynamic diameter. The droplet size distribution is reproducible. When administering proteins or other immunogenic compounds, minimizing lung exposure may be an important consideration in avoiding a hypersensitive immune response.

Between uses, apparatus (12) rests on base (5) in an upright position. Apparatus (12) possesses a low center of gravity so that it does not topple over easily, and it restores itself to an upright position when oriented within 15 degrees of an upright position and released on a horizontal surface. The low center of gravity is an advantageous property during both device manufacture and patient use.

### Other Embodiments

Other embodiments are within the following claims. For example, the vial of the invention may contain a well of any geometric shape, including conical, pyramidal, semispherical, or cylindrical. The well may be truncated, or it may taper to a point. It is also possible for the vial to lack any well at its base. Additionally, the vial of the invention may possess a non-circular cross-section. For example, the cross-section could be a triangle, square, rectangle, trapezoid, parallelogram, ellipse, or any other regular or non-regular polygon or other closed two-dimensional geometric shape. Furthermore, the vial may be composed of any substance, including glass, plastic, or other materials. The vial may be any color, including gray, white, or brown; use of a brown vial may help minimize oxidative degradation of the medication. The vial may be colorless. It may also be transparent, translucent, or opaque.

The apparatus of the invention need not have a low center of gravity, and it need not be designed to remain upright when set down on a horizontal surface.

The dip tube of the invention may have a notch of any shape, or it may lack a notch altogether. Alternatively, the dip tube may have a diagonal slanted end. The dip tube may be positioned such that it is in direct contact with the vial, or it may be positioned not to be in contact with the vial.

The vial of the invention may be very small, so that it constitutes a monodose system.

The apparatus may be used to treat non-oral disorders. For example, the apparatus may be used to treat corneal lesions in the eye, keratoconjunctivitis sicca (dry eye), or other ophthalmic disorders or injuries. It may also be used following refractive surgery. Other regions of the body that may be treated include the ear, the nasal passages, the respiratory tract (including the lungs, with modification of the medication droplet size), the extremities (i.e. hands and feet), any part of the skin, the anus, or the genitalia or genitourinary tract, including vaginal, cervical, or uterine mucosa. Any sort of wound, such as a lesion, an ulcer, a burn, or an abrasion, may be treated with the invention. Any epithelial cells of the body may be treated with the invention. Any respiratory condition, such as asthma, may be treated with the invention. Additionally, internal wounds or injuries may be treated with the invention, for example during a surgical procedure.

The apparatus of the invention need not contain medication. The apparatus can contain saline solution, water, or an alcohol/water mixture. If the apparatus does contain liquid medication, it may include purified or non-purified polypeptide, small molecules, any FDA-approved drug, any non-FDA-approved drug, or any other therapeutic compound. The medication may contain an aqueous or non-aqueous solution. The medication may also be in the form of a water/oil, oil/water, or water/oil/water emulsion. Furthermore, the medicine may include liposomes; in this embodiment, actuation of the apparatus will not disrupt the lamellae of the liposomes. Additionally, the medication may include anti-inflammatory agents, antibacterial agents, antiviral agents, antifungal agents, topical antiseptics, analgesics, or steroids. Examples of these agents may be found in U.S. patent application Ser. No. 10/434,752, which is hereby incorporated by reference. The medication need not contain a mucoadhesive agent, particularly when treating disorders for which the presence of a mucoadhesive agent could reduce the therapeutic effect of such medication.

The apparatus of the invention may be assembled under ordinary (i.e. non-filtered air) conditions, and an aseptic processing step is not required.

The vial and the pump assembly of the invention may be attached by any mechanism, including screwing, crimping, or snapping into place. In addition, the vial of the invention may be closed with a separate screw cap, under sterile or nonsterile conditions; the pump assembly may then be attached at a later time, e.g., immediately before use by a patient.

The apparatus of the invention may be adjusted to modulate the volume of liquid medication administered in each actuation cycle. For example, pumps with different metering chamber volumes may be selected in order to deliver different dosages, or the pump assembly may contain an adjustable dial that controls the volume of liquid medication that is expelled. Additionally, the drug dosage per actuation may be adjusted by altering the drug concentration in the liquid medication of the apparatus.

The apparatus of the invention may include a precompression pump. Such a pump may include a spring that is partially compressed prior to actuation, so that the full metered dosage is expelled upon actuation, even if the patient depresses the actuator slowly or partially. Such a pump prevents the administration of a partial dosage of the liquid medication of the apparatus.

The apparatus of the invention may require any number of priming cycles prior to initial use. A pump specifically designed to minimize priming may be used when the medication of the invention is particularly expensive or difficult to obtain. In an alternative embodiment, no priming cycles are required.

The apparatus of the invention may be used over a wide range of operating temperatures without significantly altering the performance of the apparatus.

The apparatus of the invention may include a means for generating any desirable plume geometry; such means may be modulated to influence the size, shape, and composition of the plume of liquid medication created by actuation. For example, the size distribution of medication droplets in the plume may be modified to favor pulmonary deposition of the droplets. Specifically, an increased percentage, such as 5%, 10%, 20%, 50%, or 100% of the droplets, may be between 5 and 10 microns in aerodynamic diameter and thus more easily deposited into the bronchiolar region. In addition, an increased percentage, such as 5%, 10%, 20%, 50%, or 100% of the droplets, may be between 1 and 6 microns in aerodynamic diameter and thus more easily deposited into the alveolar regions of the lung. Furthermore, if a mucoadhesive agent is included in the medication of the invention, the plume droplet size distribution may be modified to optimize the uniformity of the spray distribution on mucosal surfaces based on the viscosity of the mucoadhesive.

One factor that influences plume geometry is the geometry of the spray arm nozzle of the invention. For example, in one embodiment, the apparatus of the invention contains a spray arm nozzle that produces a wide cone angle of spray; if this is used in combination with a spray arm of 5 cm or less, such an apparatus is particularly useful for treating disorders that causes lesions throughout the oral cavity, such as chemotherapy-induced mucositis or aphthous stomatitis. In an alternative embodiment, a different spray arm nozzle geometry results in a narrower angle of spray; if used in combination with a spray arm of 5 cm or longer, the apparatus of this embodiment is particularly useful for treating disorders that affect the soft palate or other regions deep within the oral cavity or esophagus. For example, radiation therapy administered to the head and neck could cause such a disorder.

The apparatus of the invention may be used as a single-dose or multi-dose delivery system. For example, the apparatus may be used once per day, 2-40 times per day, or any other desirable frequency of administration, including any desirable number of actuations per administration.

The methods of the invention may feature self-administration using the apparatus of the invention; alternatively, another person, such as a medical care provider, can administer medication to a patient using such apparatus, or administration can be effected in an automated fashion featuring actuation via sound, a timer, or any other means.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A liquid medication delivery apparatus, said apparatus comprising:
a) a vial for containing said liquid medication; and
b) a pump assembly detachably attached to said vial, said pump assembly comprising:
i) a dip tube having an opening therethrough, said dip tube positioned inside said vial;
ii) a moveable spray arm having a nozzle; and
iii) a means for expelling said medication from said vial through said opening in said dip tube and out said nozzle.

2. The apparatus of claim 1, said vial comprising an inner well at its base in which said medication may accumulate, said opening in said dip tube positioned inside said well.

3. The apparatus of claim 2, wherein said well is in the shape of a frustum.

4. The apparatus of claim 1, wherein said apparatus restores itself to an upright position when oriented within 15 degrees of an upright position and released on a horizontal surface.

5. The apparatus of claim 1, wherein said opening in said dip tube comprises a notch.

6. The apparatus of claim 5, wherein said notch is v-shaped.

7. The apparatus of claim 1, wherein said apparatus is used to treat a disorder of the oral cavity.

8. The apparatus of claim 7, wherein said disorder comprises mucositis.

9. The apparatus of claim 7, wherein said disorder comprises stomatitis.

10. The apparatus of claim 9, wherein said stomatitis is aphthous stomatitis.

11. The apparatus of claim 7, wherein said disorder is induced by antineoplastic therapy.

12. The apparatus of claim 7, wherein said disorder comprises candidiasis.

13. The apparatus of claim 7, wherein said disorder comprises gingivitis.

14. The apparatus of claim 7, wherein said disorder comprises periocoronitis.

15. The apparatus of claim 7, wherein said disorder comprises periodontitis.

16. The apparatus of claim 7, wherein said disorder comprises xerostomia.

17. The apparatus of claim 1, wherein said apparatus additionally comprises said liquid medication.

18. The apparatus of claim 17, wherein said medication comprises purified polypeptide.

19. The apparatus of claim 18, wherein said medication comprises a trefoil peptide.

20. The apparatus of claim 19, wherein said trefoil peptide is ITF (intestinal trefoil factor).

21. The apparatus of claim 20, wherein said ITF is human ITF.

22. The apparatus of claim 19, wherein said trefoil peptide is an analog or biologically active fragment of ITF.

23. The apparatus of claim 22, wherein said biologically active fragment of ITF is selected from the group consisting of hITF₁₅₋₇₃, hITF₂₅₋₆₂, hITF₂₂₋₆₂, hITF₂₁₋₆₂, hITF₂₅₋₇₀, hITF₂₂₋₇₀, hITF₂₅₋₇₂, hITF₂₂₋₇₂, hITF₂₁₋₇₂, hITF₂₅₋₇₃, hITF₂₂₋₇₃, hITF₂₁₋₇₃, and EA-hITF₁₅₋₇₃.

24. The apparatus of claim 19, wherein said trefoil peptide is spasmolytic polypeptide (SP) or pS2.

25. The apparatus of claim 19, wherein said trefoil peptide is an analog or biologically active fragment of spasmolytic polypeptide or pS2.

26. The apparatus of claim 1, wherein said means for expelling said medication comprises a spray actuator.

27. The apparatus of claim 1, wherein said means for expelling said medication comprises an electronic component.

28. The apparatus of claim 17, wherein deployment of said pump assembly causes a therapeutically effective dosage of said medication to be expelled through said nozzle.

29. The apparatus of claim 17, wherein said pump, once primed, remains primed, provided that said opening in said dip tube remains immersed in said medication.

30. The apparatus of claim 1, wherein said moveable spray arm is shorter than 5 centimeters (cm).

31. The apparatus of claim 1, wherein said moveable spray arm is 5 cm or longer.

32. The apparatus of claim 1, wherein said vial is compatible with more than one pump.

33. The apparatus of claim 32, wherein the volume of said medication expelled in each actuation may be changed by detaching one pump assembly and attaching another pump assembly.

34. The apparatus of claim 1, wherein said apparatus requires less than 100 Newtons of force to actuate.

35. The apparatus of claim 17, wherein said apparatus, upon actuation, produces a spray of said medication comprising droplets, wherein no greater than 1% of said droplets are smaller than 10 microns in aerodynamic diameter.

36. The apparatus of claim 17, wherein said medication consists of a solution, suspension, or emulsion.

37. The apparatus of claim 17, wherein said medication comprises a mucoadhesive agent.

38. The apparatus of claim 37, wherein said mucoadhesive agent is selected from the group consisting of a Carbopol, a pectin, an alginate, a chitosan, an oligosaccharide, a polysaccharide, a gellan, a carrageenan, a xanthan gum, gum Arabic, a Tamarind gum, a dextran, a cellulose, a derivative of cellulose, a polyether polymer, a polyether oligomer, a fatty acid, a fatty alcohol, a fatty amide, a polyhydric alcohol, a polyether compound, a polyacrylamide, a poly(vinyl pyrrolidone), a poly(methacrylic acid), a poly(acrylic acid), and a Carbomer.

39. The apparatus of claim 37, wherein said medication additionally comprises a surfactant.

40. The apparatus of claim 39, wherein said surfactant comprises a sorbitan ester.

41. The apparatus of claim 1, wherein said apparatus additionally comprises materials that have low sorption capability.

42. The apparatus of claim 17, wherein said medication comprises an antimicrobial preservative.

43. The apparatus of claim 1, wherein said vial comprises glass.

44. The apparatus of claim 1, wherein said vial comprises plastic.

45. A liquid medication delivery apparatus, said apparatus comprising:
a) a vial for containing said liquid medication; and
b) a pump assembly detachably attached to said vial, said pump assembly comprising:
i) an intake opening, said opening positioned below said vial;
ii) a moveable spray arm having a nozzle; and
iii) a means for expelling said medication from said vial into said intake opening and out said nozzle.

46. A method of treating a patient, said method comprising using the apparatus of claim 1 or claim 45.

47. The method of claim 46, wherein said apparatus is used to treat an oral disorder in said patient.

48. The method of claim 47, wherein said disorder comprises mucositis.

49. The method of claim 47, wherein said disorder comprises stomatitis.

50. The method of claim 49, wherein said stomatitis is aphthous stomatitis.

51. The method of claim 47, wherein said disorder is induced by antineoplastic therapy.

52. The method of claim 47, wherein said disorder comprises candidiasis.

53. The method of claim 47, wherein said disorder comprises gingivitis.

54. The method of claim 47, wherein said disorder comprises periocoronitis.

55. The method of claim 47, wherein said disorder comprises periodontitis.

56. The method of claim 47, wherein said disorder comprises xerostomia.

57. The method of claim 46, wherein said apparatus comprises said liquid medication.

58. The method of claim 57, wherein said medication comprises purified polypeptide.

59. The method of claim 58, wherein said medication comprises a trefoil peptide.

60. The method of claim 59, wherein said trefoil peptide is ITF (intestinal trefoil factor).

61. The method of claim 60, wherein said ITF is human ITF.

62. The method of claim 59, wherein said trefoil peptide is an analog or biologically active fragment of ITF.

63. The method of claim 62, wherein said biologically active fragment of ITF is selected from the group consisting of hITF₁₅₋₇₃, hITF₂₅₋₆₂, hITF₂₂₋₆₂, hITF₂₁₋₆₂, hITF₂₅₋₇₀, hITF₂₂₋₇₀, hITF₂₅₋₇₂, hITF₂₂₋₇₂, hITF₂₁₋₇₂, hITF₂₅₋₇₃, hITF₂₂₋₇₃, hITF₂₁₋₇₃, and EA-hITF₁₅₋₇₃.

64. The method of claim 59, wherein said trefoil peptide is spasmolytic polypeptide (SP) or pS2.

65. The method of claim 59, wherein said trefoil peptide is an analog or biologically active fragment of spasmolytic polypeptide or pS2.

66. The apparatus of claim 7, wherein said disorder comprises halitosis.
